SEQUENCE LISTING (25 sequences)

SEQ ID NO: 1 - protein, 10 aa, synthetic construct
ELGRECLNLW

SEQ ID NO: 2 - protein, 13 aa, synthetic construct
RCMELGRECLNLW

SEQ ID NO: 3 - protein, 21 aa, synthetic construct
RRRRRRRRRCMELGRECLNLW

SEQ ID NO: 4 - protein, 18 aa, synthetic construct
QLQRIIRTGRTGHWLNHG

SEQ ID NO: 5 - protein, 8 aa, synthetic construct
FGRPHNVQ

SEQ ID NO: 6 - protein, 18 aa, synthetic construct
GRAMELGRECLNLWGYER

SEQ ID NO: 7 - protein, 13 aa, synthetic construct
RAMELGRECLNLW

SEQ ID NO: 8 - protein, 22 aa, synthetic construct
RRRRRRRRRRAMELGRECLNLW

SEQ ID NO: 9 - protein, 22 aa, synthetic construct
RRRRRRRRRRKMELGREFLNLW

SEQ ID NO: 10 - protein, 22 aa, synthetic construct
RRRRRRRRRRKMELGREYLNLW

SEQ ID NO: 11 - protein, 22 aa, synthetic construct
RRRRRRRRRRKMELGREMLNLW

SEQ ID NO: 12 - protein, 22 aa, synthetic construct
RRRRRRRRRRKMELGRELLNLW

SEQ ID NO: 13 - protein, 22 aa, synthetic construct
RRRRRRRRRRRMELGRELLNLW

SEQ ID NO: 14 - protein, 22 aa, synthetic construct
RRRRRRRRRRKWELGRELLNLW

SEQ ID NO: 15 - protein, 22 aa, synthetic construct
RRRRRRRRRRAMELGRECLNLA

SEQ ID NO: 16 - protein, 22 aa, synthetic construct
RRRRRRRRRRAMEEGRECLNLW

SEQ ID NO: 17 - protein, 22 aa, synthetic construct
RRRRRRRRRRKWCLGRELLCLW

SEQ ID NO: 18 - protein, 22 aa, synthetic construct
RRRRRRRRRRKWCLGRELLCLW

SEQ ID NO: 19 - protein, 7 aa, synthetic construct
NAPFFKY

SEQ ID NO: 20 - protein, 4 aa, synthetic construct
GYYF

SEQ ID NO: 21 - protein, 7 aa, synthetic construct
KLVFFAE

SEQ ID NO: 22 - protein, 13 aa, synthetic construct
GRKKRRQRRRPPQ

SEQ ID NO: 23 - protein, 16 aa, synthetic construct
RQIKIWFQNRRMKWKK

SEQ ID NO: 24 - protein, 18 aa, synthetic construct
KLALKLALKALKAALKLA

SEQ ID NO: 25 - protein, 26 aa, synthetic construct
GIGAVLKVLTTGLPALISWIKRKRQQ

(19) United States
(12) Patent Application Publication
(10) Pub. No.: US 2025/0296956 A1
(43) Pub. Date: Sep. 25, 2025

(54) POLYPEPTIDE, PREPARATION METHOD, AND USE THEREOF

(71) Applicants: Hunan University, Changsha (CN); GREATER BAY AREA INSTITUTE FOR INNOVATION, HUNAN UNIVERSITY, Guangzhou (CN)

(72) Inventors: Junfeng Shi, Changsha (CN); Dingxiao Zhang, Changsha (CN); Zenghui Li, Changsha (CN); Yuqing Feng, Changsha (CN); Mingshui Wang, Changsha (CN); Hong Han, Changsha (CN)

(21) Appl. No.: 19/066,205

(22) Filed: Feb. 28, 2025

Related U.S. Application Data
(63) Continuation of application No. PCT/CN2023/116629, filed on Sep. 1, 2023.

(30) Foreign Application Priority Data
Sep. 9, 2022 (CN) 202211104956.7
Sep. 9, 2022 (CN) 202211105358.1

(51) Int. Cl. C07K 7/08; A61K 9/00; A61K 9/08; A61K 38/00; A61P 35/00; C07K 7/06

(52) U.S. Cl. CPC C07K 7/08; A61K 9/0019; A61K 9/08; A61P 35/00; C07K 7/06; A61K 38/00; C07K 2319/10

(57) ABSTRACT

The present disclosure relates to a biochemical field, and provides a polypeptide, the polypeptide includes: (I), an amino acid sequence as shown in any one of SEQ ID No.1, SEQ ID No.4, and SEQ ID No.5; or (II), an amino acid sequence obtained by substituting, deleting, or adding one or more amino acids to the amino acid sequence of (I), and having a same function as the amino acid sequence of (I); or (III) an amino acid sequence with greater than 80% similarity to the amino acid sequence of (I) or (II); or (IV) a cell-penetrating polypeptide obtained by combining the polypeptide of (I), (II), or (III) with a cell-penetrating peptide; or (V) a stapled peptide obtained by stapling the polypeptide of (I), (II), or (IV). The polypeptide has a significant inhibitory effect on methylation and METTL3 expression. A preparation method and use of the polypeptide are further provided.

### CROSS-REFERENCE TO RELATED APPLICATIONS

The present application is a continuation application of PCT application No. PCT/CN2023/116629 filed on Sep. 1, 2023, which claims the benefit of Chinese Patent Application No. 202211104956.7 filed on Sep. 9, 2022 and Chinese Patent Application No. 202211105358.1 filed on Sep. 9, 2022. The contents of all of the aforementioned applications are incorporated by reference herein in their entirety.

### REFERENCE TO SEQUENCE LISTING

The present application includes a Sequence Listing filed electronically as an XML file named “Sequence listing_SHSIN-25002-USPT.xml”, created on Jun. 13, 2025, with a size of 22,882 bytes. The sequence Listing is incorporated herein by reference.

### TECHNICAL FIELD

The present disclosure relates to a biochemical field, in particular to a polypeptide derivative, a preparation method, and use thereof.

### BACKGROUND

Prostate cancer (PCa) is one of the most common malignant tumors in males, and is the second leading cause of deaths in males. According to statistics, the incidence and mortality rates of the prostate cancer are increasing year by year, with over 300,000 deaths annually due to PCa, accounting for 6.6% of all deaths. Currently, androgen deprivation therapy (ADT) is one of the common treatment methods for PCa, in addition to surgery, ADT works by interfering with androgen/androgen receptor signaling pathway using androgen synthesis inhibitors (e.g., abiraterone) or androgen receptor inhibitors (e.g., bicalutamide, flutamide, enzalutamide, etc.) to inhibit the growth of cancer cells. ADT is effective in early stage of cancer, but is prone to drug resistance, typically, after 3 to 5 years of treatment, the disease progresses to castration-resistant prostate cancer (CRPC), with a 5-year survival rate of only 25.4%. Currently, the FDA has not approved any effective drugs for treating CRPC, and the approved prostate cancer drugs have limited efficacy, only extending lives of the patients by a few months. Therefore, it is extremely urgent and necessary to conduct in-depth research on new drugs for PCa.

A website https://wenku.baidu.com/view/8349065a940590c69ec3d5bbfd0a79563d1ed41d.html documents a polypeptide-based DNA methyltransferase inhibitor-DNA methylation inhibitor, the inhibitor disclose 5-azacytidine, PEP515, bovine lactoferrin, and HDAC cyclic peptide inhibitor.

It can be seen that, polypeptides as methylation inhibitors have already begun to be studied by numerous research institutions.

Polypeptides suitable for methylation inhibition are not limited to the above examples.

Therefore, the technical problem to be solved in the present disclosure is that how to develop new polypeptides that inhibit methyltransferase activity and m6A modification, as well as how to develop a new polypeptide-based anti-tumor drug and how to inhibit the growth and proliferation of tumor cells.

### SUMMARY

A purpose of the present disclosure is to provide a polypeptide including a number of selectable forms and having a relatively obvious RNA methylation and METTL3 expression inhibitory effect.

At the same time, the present disclosure further discloses a preparation method and use of the polypeptide.

Unless otherwise specified in the present disclosure: mM represents millimole/liter, μM represents micromole/liter, nM represents for nanomoles per liter.

To achieve the above purpose, the present disclosure provides the following technical solution: a polypeptide includes:

(I), an amino acid sequence shown in SEQ ID No.1;

a sequence of SEQ ID No. 1 is:
ELGRECLNLW;
a sequence of SEQ ID No. 4 is:
QLQRIIRTGRTGHWLNHG;
a sequence of SEQ ID No.5 is:
FGRPHNVQ;

the above-mentioned polypeptide sequences SEQ ID No. 4 & 5 are remaining two polypeptide screening sequences that can inhibit METTL3;

or

(II), an amino acid sequence obtained by substituting, deleting, or adding one or more amino acids to the amino acid sequence described of (I), and having a same function as the amino acid sequence of (I);

(III), an amino acid sequence with greater than 80% similarity to the amino acid sequence

of (I) or (II);

for example: SEQ ID No. 6:
GRAMELGRECLNLWGYER;

the sequence is obtained by each extending 4 amino acids forward and backward upon ELGRECLNLW;

for example, SEQ ID No. 2:
RCMELGRECLNLW;

the sequence is obtained by mutating the A amino acid of SEQ ID No.7 to C;

for example, SEQ ID No. 7:
RAMELGRECLNLW;

the sequence is obtained by extending forward by 3 amino acids upon SEQ ID No. 1: ELGRECLNLW; the sequence is a protein truncated peptide of METTL3 like M3 peptide, and has similar functions to M3 peptide, this part is the functional region of the target polypeptide drug, which can bind to METTL3 and METTL14 complex proteins and exert the effect;

when the sequence is put into use, the sequence can be combined with various membrane-penetrating peptide structures, such as R9, to obtain R9-MPF13, whose amino acid sequence is: SEQ ID No. 8: RRRRRRRRRRAMELGRECLNLW;

The sequence can be further modified on the basis of R9-MPF13 by replacing some amino acids in the functional region. The enhancing mutations are concentrated in the 11th, 12th, and 18th positions of the amino acid sequence, such as the polypeptides R9-RKF, R9-RKY, R9-RKM, R9-RKL, R9-RRL, and R9-RKWL, this type of mutation can enhance the anti-proliferative effect of the polypeptide; the weakening mutations are concentrated in the 14th and 22nd positions of the amino acid sequence, such as the polypeptides R9-RA and R9-RE, this type of mutation can weaken the anti-proliferative effect of the polypeptide. The polypeptide R9-PSRKWL is obtained based on R9-RKWL, and the amino acids at positions 13 and 20 of the amino acid sequence of R9-RKWL are mutated to cysteine, which is a stapled peptide precursor. R9-SRKWL is a stapled peptide obtained by stapling R9-PSRKWL at the 13th and 20th cysteines of the polypeptide via 4,4-bis(bromomethyl)biphenyl connection.

The polypeptide sequences are listed in Table 1 below:

TABLE 1
Sequence Listing

| Polypeptide name | sequence | SEQ ID No |
|---|---|---|
| M3 | ELGRECLNLW | 1 |
| M3-1 | RCMELGRECLNLW | 2 |
| RM3 | RRRRRRRRRCMELGRECLNLW | 3 |
| M1 | QLQRIIRTGRTGHWLNHG | 4 |
| M2 | FGRPHNVQ | 5 |
| M3-2 | GRAMELGRECLNLWGYER | 6 |
| MPF13 | RAMELGRECLNLW | 7 |
| R9-MPF13 | RRRRRRRRRRAMELGRECLNLW | 8 |
| R9-RKF | RRRRRRRRRRKMELGREFLNLW | 9 |
| R9-RKY | RRRRRRRRRRKMELGREYLNLW | 10 |
| R9-RKM | RRRRRRRRRRKMELGREMLNLW | 11 |
| R9-RKL | RRRRRRRRRRKMELGRELLNLW | 12 |
| R9-RRL | RRRRRRRRRRRMELGRELLNLW | 13 |
| R9-RKWL | RRRRRRRRRRKWELGRELLNLW | 14 |
| R9-RA | RRRRRRRRRRAMELGRECLNLA | 15 |
| R9-RE | RRRRRRRRRRAMEEGRECLNLW | 16 |
| R9-PSRKWL | RRRRRRRRRRKWCLGRELLCLW | 17 |
| R9-SRKWL(staple | RRRRRRRRRRKWCLGRELLCLW | 18 |
| peptide, between two | | |
| Cs) | | |
| | NapFFKY | 19 |
| | GYYF | 20 |
| | KLVFFAE | 21 |
| | GRKKRRQRRRPPQ | 22 |
| | RQIKIWFQNRRMKWKK | 23 |
| | KLALKLALKALKAALKLA | 24 |
| | GIGAVLKVLTTGLPALISWIKRKRQQ | 25 |

(Note: The polypeptide is N to C terminus from left to right); or (IV), a cell-penetrating polypeptide obtained by combining the polypeptide shown in (I), (II) or (III) with a cell-penetrating peptide; or (V), a stapled peptide obtained by stapling the polypeptide shown in (I), (II) or (IV).

The sequence shown in SEQ ID No.1 has at least the following variations:

1. The sequence can combine with self-assembling polypeptides, such as SEQ ID No. 19: NapFFKY and SEQ ID No. 20: GYYF, SEQ ID No. 21: KLVFFAE (core sequence in amyloid protein Aβ), to develop new polypeptide drugs.

2. The sequence can be a stapled peptide obtained by stapling the polypeptide with the amino acid sequence as shown in any one of SEQ ID No.1, SEQ ID No.4, and SEQ ID No.5, or a stapled peptide obtained by replacing at least one amino acid in the amino acid sequence as shown in SEQ ID No.1 with a reactive side chain non-natural amino acid.

For SEQ ID No. 1, any form of stapled peptide construction can be performed on the sequence through other linking molecules, such as: replacing E at position 1 and E at position 5, and N at position 8, as well as R, E, and N with reactive side chain non-natural amino acids.

3. The sequence can be added or replace with non-natural amino acids, such as changing the amino acid from L type to D type.

4. Modify polypeptide drugs through chemical modification, such as use PEG modification to increase drug circulation time; use fatty acid (such as C12 or C18) modification to increase stability and utilization.

5. Deliver the polypeptide drug by combining the polypeptide with a drug carrier, such as liposomes, microspheres, micelles, and hydrogels, to prepare a new drug dosage form.

In the above-mentioned polypeptides, the cell-penetrating peptide is a cell-penetrating peptide that can be retrieved by CPPsite 2.0 (https://webs.iiitd.edu.in/raghava/cppsite/stats1.php), such as one of several classic cell-penetrating peptides: R9 cell-penetrating peptide, TAT cell-penetrating peptide (SEQ ID No. 22: GRKKRRQRRRPPQ), Penetratin cell-penetrating peptide (SEQ ID No. 23: RQIKIWFQNRRMKWKK) MAP (SEQ ID No. 24: KLALKLALKALKAALKLA), Melittin (SEQ ID No. 25: GIGAVLKVLTTGLPALISWIKRKRQQ).

In the above polypeptide, the polypeptide has an amino acid sequence as shown in SEQ ID No.2.

The sequence of SEQ ID No. 2 is:
RCMELGRECLNLW.

In the above-mentioned polypeptide, the second and ninth cysteines of the polypeptide with the amino acid sequence shown in SEQ ID No. 2 are stapled by 4,4-bis(bromomethyl)biphenyl to obtain a stapled peptide named RSM3.

In the above polypeptide, the membrane-penetrating polypeptide includes an amino acid sequence as shown in SEQ ID No.3.

The sequence of SEQ ID No.3 is:
RRRRRRRRRCMELGRECLNLW.

In the above polypeptide, the 10th and 17th cysteines of the polypeptide with the amino acid sequence shown in SEQ ID No. 3 are connected through 4,4-bis(bromomethyl)biphenyl to construct the i+7 stapled peptide.

At the same time, the present disclosure further provides a preparation method of the polypeptide as described above, the preparation method includes calculating and weighing required amino acids, dissolving the amino acids using AM resin, placing the amino acids in a synthesis bottle, setting up and synthesizing the amino acids according to operations of a fully automatic synthesizer, taking out the AM resin, obtaining the polypeptide by cutting and precipitating, and purifying the polypeptide.

In addition, the present disclosure further provides use of the polypeptide, the polypeptide is used to prepare a drug for treating cancer, or the polypeptide is used to inhibit the growth and/or proliferation of tumor cells, more preferably, the polypeptide is used to inhibit the growth and/or proliferation of tumor cells in vitro, that is, as an inhibitor of the growth and/or proliferation of cells separated from the human body in a laboratory.

As a preferred embodiment of the present disclosure, the drug is used to treat one or more of liver cancer, prostate cancer, thyroid tumors, leukemia, pancreatic cancer, colorectal cancer, lung cancer, glioblastoma, breast cancer, bladder cancer, gastric cancer, pancreatic cancer, osteosarcoma, oral squamous cell carcinoma, melanoma, ovarian cancer, head and neck squamous cell carcinoma, skin squamous cell carcinoma and nasopharyngeal carcinoma.

In the above-mentioned use of the polypeptide, the dosage form of the drug is an injection or an oral preparation;

the drug is a water solution or hydrogel dispersed with the polypeptide, or the drug is a liposome, microsphere capsule, or micelle encapsulating the polypeptide, or the polypeptide constitutes the liposome, the microsphere capsule, or the micelle to form the drug in a form of shell.

In the above-mentioned use of the polypeptide, the oral preparation is a pill, tablet, capsule or granule.

In the above-mentioned use of the polypeptide, the tumor cells are one or more of human osteosarcoma cells, human hepatoblastoma cells, human prostate cancer cells, colorectal cancer cells, human malignant melanoma, human cervical cancer cells, human alveolar basal epithelial cells, human chronic myeloid leukemia cells, and ovarian adenocarcinoma cells.

Compared with the prior art, the present disclosure has the following beneficial effects:

the polypeptide inhibitor provided in the present disclosure has a function of inhibiting RNA methyltransferase METTL3, and enables the polypeptide drug to enter cells to exert its effect by binding to the cell-penetrating peptide R9;

by synthesizing staple peptides on the original sequence of the polypeptide, the structural stability of the polypeptide is increased, and the activity and efficacy of the polypeptide drug are improved;

the polypeptide drug can specifically bind to METTL3 and inhibit the activity of RNA

methyltransferase, thereby reducing the RNA methylation level;

the polypeptide can significantly inhibit the growth and proliferation of SaoS2 (human osteosarcoma cells), HepG2 (human hepatoblastoma cells), DU145 (human prostate cancer cells), HCT116 (colorectal cancer cell line), A375 (human malignant melanoma), Hela (human precervical cancer cells), A549 (lung cancer human alveolar basal epithelial cells), K562 (human chronic myeloid leukemia cells), and OVCAR-3 (ovarian adenocarcinoma cells).

### BRIEF DESCRIPTION OF THE DRAWINGS

FIG. 1 is a structural view of a polypeptide sequence and molecular structure provided in Embodiment 1 according to the present disclosure;

FIG. 2 illustrates synthesis steps of a stapled peptide provided in Embodiment 2 according to the present disclosure;

FIG. 3 is a schematic view of RNA methylation inhibition effect on prostate cancer cells provided by Embodiment 3 according to the present disclosure;

FIG. 4 is a schematic view of results of inhibiting expression of METTL3 on prostate cancer cells provided in Embodiment 4 according to the present disclosure;

FIG. 5A is a schematic view of growth inhibition results of prostate cancer cells provided by Embodiment 5 according to the present disclosure;

FIG. 5B is a schematic view of effect of inhibiting growth of prostate cancer cells provided by Embodiment 5 according to the present disclosure;

FIG. 6 is a schematic view of growth inhibition results of prostate cancer cells provided by Embodiment 6 according to the present disclosure;

FIG. 7A is a schematic view of indexes of white blood cells in blood of mice after polypeptides R9, RM3 and RSM3 were injected into the body according to the present disclosure;

FIG. 7B is a schematic view of indexes of platelet in blood of mice after the polypeptides R9, RM3 and RSM3 were injected into the body according to the present disclosure;

FIG. 7C is a schematic view of indexes of red blood cells in blood of mice after the polypeptides R9, RM3 and RSM3 were injected into the body according to the present disclosure;

FIG. 7D is a schematic view of indexes of hemoglobin in blood of mice after the polypeptides R9, RM3 and RSM3 were injected into the body according to the present disclosure;

FIG. 7E is a schematic view of changes in body weight of tumor model mice after the polypeptides R9, RM3 and RSM3 were injected into the mice according to the present disclosure;

FIG. 7F is a schematic view of changes in tumor volume after the polypeptides R9, RM3 and RSM3 were injected into tumor model mice according to the present disclosure;

FIG. 7G is a schematic view of changes in tumor mass after the polypeptides R9, RM3 and RSM3 were injected into tumor model mice according to the present disclosure;

FIG. 7H is a schematic view of changes in tumor images after the polypeptides R9, RM3 and RSM3 were injected into tumor model mice according to the present disclosure;

FIG. 8A is a data view of survival rates of prostate cancer cell DU145 treated with various polypeptides of Embodiment 5 according to the present disclosure, in FIG. 8A, the horizontal axis represents polypeptide concentration, and the vertical axis represents cell survival rate;

FIG. 8B illustrates a table of inhibitory concentrations of various polypeptides of Embodiment 5 on prostate cancer cells DU145 according to the present disclosure, the vertical axis IC50 is half inhibitory concentration;

FIG. 9A is a data view of survival rates of various cancer cells treated with the polypeptide R9-RKL of Embodiment 5 according to the present disclosure, in FIG. 9A, the horizontal axis represents the polypeptide concentration, and the vertical axis represents the cell survival rate;

FIG. 9B is a table of inhibitory concentrations of the polypeptide R9-RKL of Embodiment 5 on various cancer cells according to the present disclosure, the vertical axis IC50 is the half inhibitory concentration.

### DETAILED DESCRIPTION

The following will be combined with the drawings in the embodiments of the present disclosure to clearly and completely describe the technical solutions in the embodiments of the present disclosure, obviously, the described embodiments are only part of the embodiments of the present disclosure, not all of the embodiments. Based on the embodiments of the present disclosure, all other embodiments obtained by the persons skilled in the art without creative work are within the scope of protection of the present disclosure.

### Embodiment 1

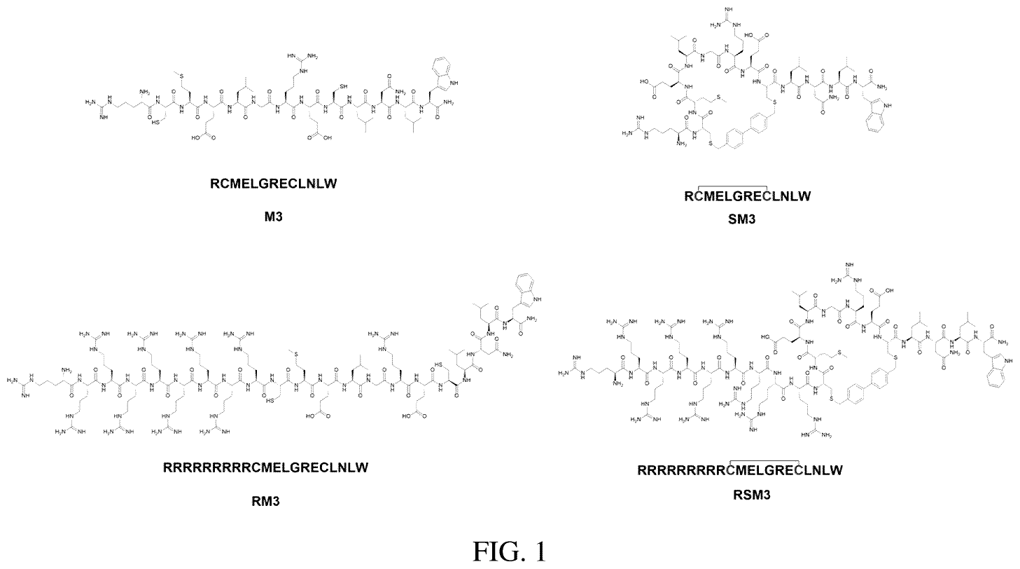

1.1 The polypeptide drug molecules were synthesized by solid phase synthesis, and the amino acid sequences were M3-1 (RCMELGRECLNLW) and RM3 (RRRRRRRRRCMELGRECLNLW) (shown in FIG. 1).

The required amino acids are weighed and dissolved using AM resin, and then placed in a synthesis bottle, synthesis is set up according to operations of a fully automatic synthesis instrument, the crude polypeptide is obtained after the resin is cut and precipitated. The corresponding polypeptide drug lyophilized powder can be obtained by purification and freeze-drying using a high performance liquid chromatography.

The importance of M3 lies in that: M3 is a core active polypeptide, M3 can bind to the proteins of METTL3 and METTL14 in vitro, the sequence cannot enter the cell to exert its effect without the membrane-penetrating peptide, therefore, RM3 has more application value in actual treatment processes.

1.2 The polypeptide drug molecules were synthesized by solid phase synthesis, and the polypeptide drug molecules were R9-MPF13 (R9-Mett13's peptide fragment) and mutant polypeptide derivatives of R9-MPF13, the polypeptide derivatives were products obtained by replacing several amino acids in R9-MPF13, the amino acid sequences were referenced in Table 1 and Table 2 below; Table 2 marked the amino acid mutation and replacement methods by bold and diagonal lines.

The required amino acids are weighed and dissolved using AM resin, and then placed in a synthesis bottle, synthesis is set up according to operations of a fully automatic synthesis instrument, the crude polypeptide is obtained after the resin is cut and precipitated. The corresponding polypeptide drug lyophilized powder can be obtained by purification and freeze-drying using a high performance liquid chromatography.

The target amino acid sequence includes two parts, a first part is the membrane-penetrating peptide R9 sequence, which provides a membrane-penetrating effect for the polypeptide drug and ensures that the functional sequence can enter the cell to exert its effect; a second part is the functional peptide MPF13 and its polypeptide derivatives, which are the functional regions of the target polypeptide drug and can bind to the METTL3 and METTL14 complex proteins and exert their effects.

1.3 The polypeptide drug molecules were synthesized by solid phase synthesis, and the polypeptide drug molecules were R9-RKL (R9-RKMELGRELLNLW), the required amino acids are weighed and dissolved using AM resin, and then placed in a synthesis bottle, synthesis is set up according to operations of a fully automatic synthesis instrument, the crude polypeptide is obtained after the resin is cut and precipitated. The corresponding polypeptide drug lyophilized powder can be obtained by purification and freeze-drying using a high performance liquid chromatography.

TABLE 2
Sequence Listing

| Sequence | Sequence | | | | | | | | | | | | | | |
|---|---|---|---|---|---|---|---|---|---|---|---|---|---|---|---|
| NO | name | 1 to 9 | 10 | 11 | 12 | 13 | 14 | 15 | 16 | 17 | 18 | 19 | 20 | 21 | 22 |
| 8 | R9-MPF13 | R9 | R | A | M | E | L | G | R | E | C | L | N | L | W |
| 9 | R9-RKF | R9 | R | K | M | E | L | G | R | E | F | L | N | L | W |
| 10 | R9-RKY | R9 | R | K | M | E | L | G | R | E | Y | L | N | L | W |
| 11 | R9-RKM | R9 | R | K | M | E | L | G | R | E | M | L | N | L | W |
| 12 | R9-RKL | R9 | R | K | M | E | L | G | R | E | L | L | N | L | W |
| 13 | R9-RRL | R9 | R | R | W | E | L | G | R | E | L | L | N | L | W |
| 14 | R9-RKWL | R9 | R | K | M | E | L | G | R | E | L | L | N | L | W |
| 15 | R9-RA | R9 | R | A | M | E | L | G | R | E | C | L | N | L | A |
| 16 | R9-RE | R9 | R | A | M | E | E | G | R | E | C | L | N | L | W |
| 17 | R9-PSRKWL | R9 | R | K | W | C | L | G | R | E | L | L | C | L | W |
| 18 | R9-SRKWL | R9 | R | K | W | C′ | L | G | R | E | L | L | C′ | L | W |

Note:
(The polypeptide is N to C terminus from left to right).

### Embodiment 2

synthesis of stapled peptides SM3 and RSM3:

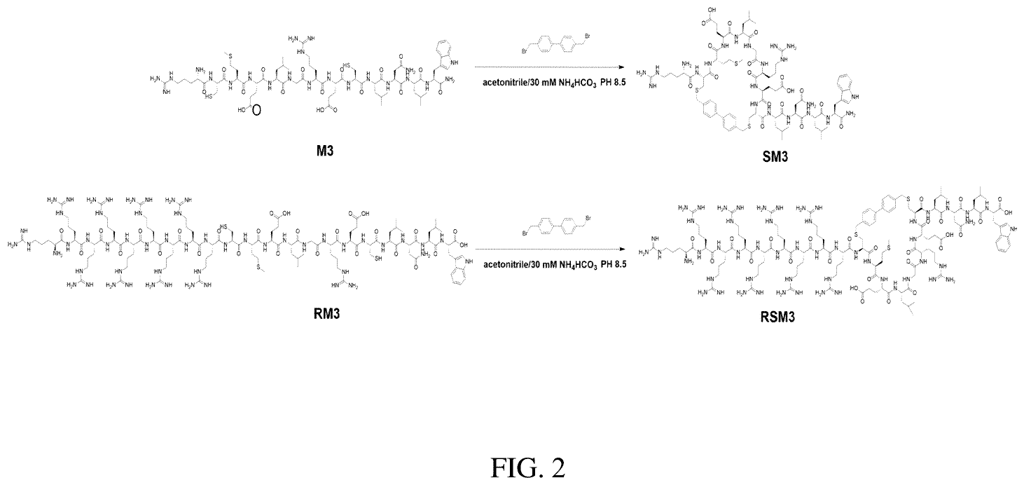

Referring to FIG. 2, the purified RM3 and M3 polypeptide freeze-dried powders were weighed and reacted respectively, and the polypeptides were dissolved in a 1:1 acetonitrile/30 mM ammonium bicarbonate mixed solution, and the final concentration of the polypeptide was 1 mM, then 1.5 mM bhp solution was added and stirred at room temperature for 3 hours. The synthesized polypeptide product was subjected to HPLC analysis and mass spectrometry detection. Finally, the stapled peptide powder was obtained after purification by high performance liquid chromatography and freeze-drying.

The stapled positions of RM3 and M3 are shown in FIG. 2, which include cysteine (Cys) at position 10 and cysteine amino acid at position 17.

### Embodiment 3

Inhibition of RNA methylation in prostate cancer cells.

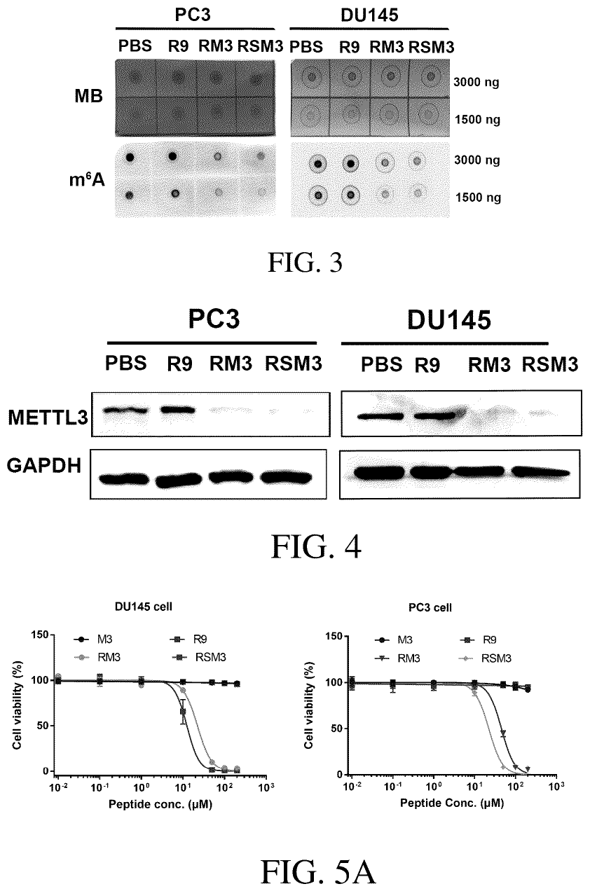

Determination of RNA methylation in cancer cells: polypeptides R9, RM3, and RSM3 were prepared into 50 mM mother solutions with sterile pure water, and then added to the 6-well plate pre-laid with PC3 and DU145 cells after gradient dilution, 2 mL per well, and three replicates for each concentration, after 12 hours, total RNA of the cells was extracted, and the RNA was added to the NC membrane prepared in advance for drying and fixation, and then incubated with primary and secondary antibodies for RNA methylation at one time, and finally exposed with ultra-sensitive ECL chemiluminescent reagent, referring to FIG. 3, the results show that RM3 and R SM3 can significantly inhibit the RNA methylation;

As illustrated in FIG. 3, the results show that: RSM3 is better in terms of stability and methylation inhibition effect when RSM3 has been stapled.

In FIG. 3, MB is methylene blue staining, and m⁶A is methylation content. 500 ng and 3000 ng are the loaded sample amounts of RNA in the experiment.

### Embodiment 4

Inhibitory effect on the expression of METTL3 in prostate cancer cells.

Referring to FIG. 4, a certain amount of polypeptide was weighed and dissolved into a 50 mM mother solution, the polypeptides R9, RM3, and RSM3 were prepared into a 50 mM mother solution with sterile pure water, after gradient dilution, the mother solution were added to a 6-well plate with PC3 and DU145 cells pre-laid, the final concentration of the polypeptide was 20 μM, 2 mL per well, and three replicates were used for each concentration. After 24 hours of treatment, the cells were lysed to extract cell proteins, and the proteins were separated by electrophoresis and transferred to a PVDF membrane, to incubate the primary antibody and secondary antibody of METTL3, and finally exposed for imaging.

As illustrated in FIG. 4, both RM3 and RSM3 were able to inhibit the expression of METTL3.

In FIG. 4, GAPDH is an internal reference in the immunoblotting experiment (WB experiment).

### Embodiment 5

Inhibitory effect on proliferation of prostate cancer cells PC3 and DU145.

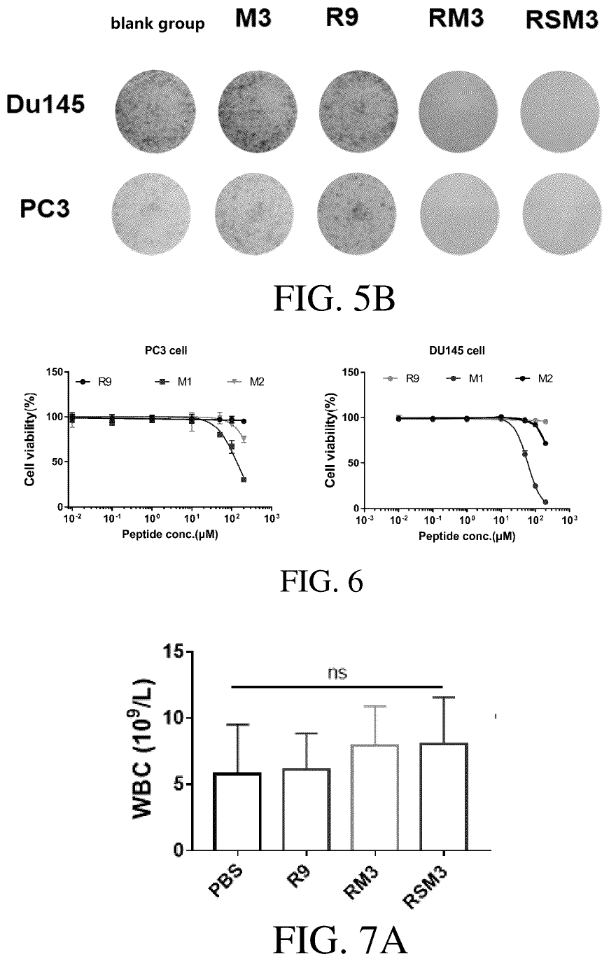

5.1 Referring to FIG. 5A, the polypeptides R9, RM3, and RSM3 were prepared into a 50 mM mother solution with sterile pure water, and the mother solution was gradiently diluted with a basal culture medium, the diluted peptide solution was added to 96-well plates pre-laid with PC3 and DU145 cells, 100 μL per well, and three replicates for each concentration. After 24 h of treatment, the OD value at 450 nm was measured with a CCK-8 kit, and the results were calculated and analyzed.

FIG. 5B illustrates a clone formation experiment, the grown PC3 and DU145 cells were digested with 0.25% trypsin and the number of cells were counted, 300 cells were added to each well of the six-well plate, and 2 ml of complete culture medium was added to continue culturing for 24 hours. M3, R9, RM3, and RSM3 were added to each well and cultured for 7 days, the medium was discarded, the cells were washed, stained with crystal violet, and then microscopically photographed, and the number of the cells were counted.

The results shows that: RSM3 and RM3 have a good inhibitory effect on the growth of prostate cancer cells, and the inhibitory effect of RSM3 is better than that of RM3.

5.2 Some of the polypeptides in Table 1 were prepared into a 10 mM mother solution with sterile pure water, and the mother solution is gradiently diluted with a basal culture medium, the diluted polypeptide solution was added to 96-well plates with DU145 cells pre-laid, 100 μL per well, and four replicates for each concentration. After 48 hours of treatment, the modified MTT kit was used to treat and measure the OD value at 580 nm, and the results were calculated and analyzed.

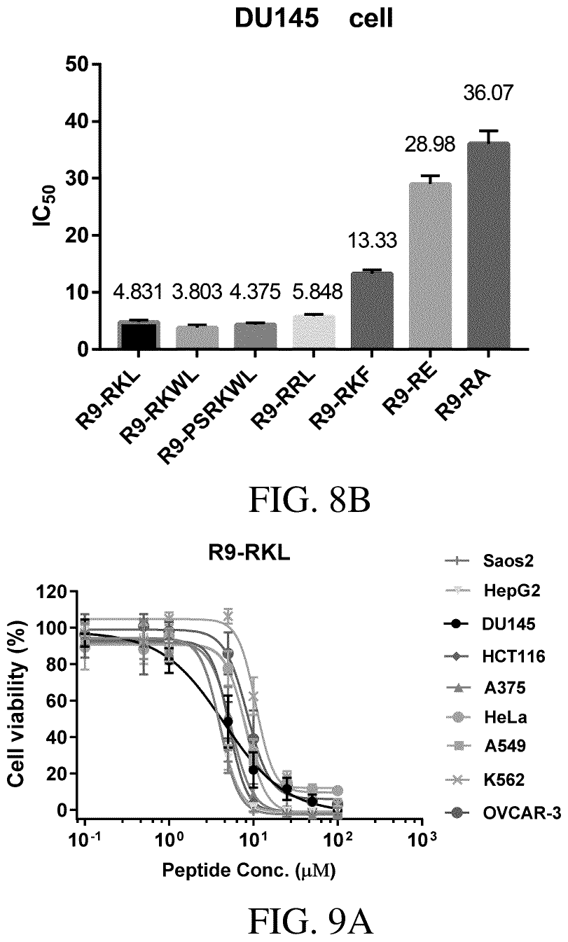

Referring to FIGS. 8A and 8B, the results show that, all polypeptides have a significant inhibitory effect on the proliferation of prostate cancer cells DU145; the three mutated polypeptides R9-RKL, R9-RKWL, and R9-PSRKWL in Table 2 have a better inhibitory effect, which indicates that the type of mutation enhances the effect of the functional peptide, and shows that the mutation of amino acid A at position 12 to K and the mutation of amino acid C at position 18 to L in the polypeptide sequence are better mutations.

FIG. 8A is a data view of survival rate of various polypeptides in Embodiment 5 of the present disclosure on prostate cancer cells DU145, in FIG. 8A, the horizontal axis is the polypeptide concentration, and the vertical axis is the survival rate of the cells.

FIG. 8B illustrates IC50 (half inhibitory concentration) of various polypeptides of Embodiment 5 of the present disclosure on prostate cancer cell DU145, in FIG. 8B, the horizontal axis is the polypeptide name, and the vertical axis is IC50.

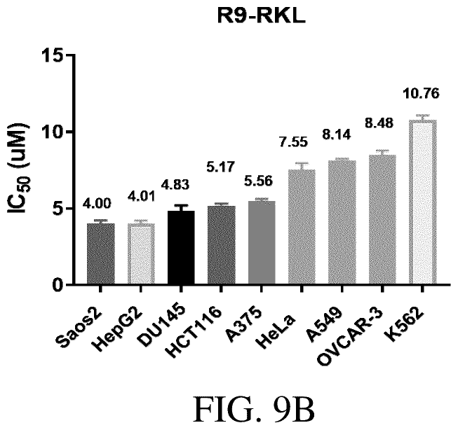

5.3 Referring to FIG. 9A and FIG. 9B; using the better effect of R9-RKL to verify the inhibitory effect on proliferation of different cancer cell lines;

The cell objects to be verified include: SaoS2 (human osteosarcoma cells), HepG2 (human hepatoblastoma cells), DU145 (human prostate cancer cells), HCT116 (colorectal cancer cell line), A375 (human malignant melanoma), Hela (human anterior cervical cancer cells), A549 (lung cancer human alveolar basal epithelial cells), K562 (human chronic myeloid leukemia cells), OVCAR-3 (ovarian adenocarcinoma cells).

The experiment method includes: the polypeptide was prepared into a 10 mM mother solution with sterile pure water, and after gradient dilution with a basal culture medium, the diluted polypeptide solution was added to a 96-well plate with various cells pre-laid, 100 μL per well, and four replicates for each concentration. After 48 hours of treatment, the modified MTT kit was used to treat and measure the OD value at 580 nm, and the results are calculated and analyzed.

FIG. 9A is a data view of the survival rate of various cells acted upon by the polypeptide R9-RKL of Embodiment 5 of the present disclosure, in FIG. 9A, the horizontal axis is the polypeptide concentration, and the vertical axis is the survival rate of the cells;

FIG. 9B is a table of inhibitory concentrations of the polypeptide R9-RKL of Embodiment 5 of the present disclosure on various cells, the vertical axis IC50 is the half inhibitory concentration.

It can be seen that from FIGS. 9A and 9B, R9-RKL has a good growth inhibitory effect on various cancer cell lines such as Sao S2, HepG2, DU145, HCT116, A375, Hela, A549, K562, and OVCAR-3, and the IC50 is lower than 11 μM.

### Embodiment 6

The other two screened sequences NO.4 & 5 M1 and M2, after being connected with the cell-penetrating peptide R9, have a certain growth inhibitory effect on prostate cancer cells PC3 and DU145.

Referring to FIG. 6, the above two polypeptides were prepared into a 50 mM mother solution with sterile pure water, and then gradiently diluted with a basal culture medium, the diluted peptide solution was added to a 96-well plate pre-laid with PC3 and DU145 cells, 100 μL per well, and three replicates for each concentration. After 24 hours of treatment, the OD value was measured at 450 nm using CCK-8, and the obtained values were calculated.

In FIG. 6, the horizontal axis represents the polypeptide concentration, and the vertical axis represents the cell survival rate.

The results showed that: M1 and M2 have a certain effect of inhibiting cell growth after combining with the cell-penetrating peptide. However, the effect is required to be further optimized and has certain research and application value.

### Embodiment 7

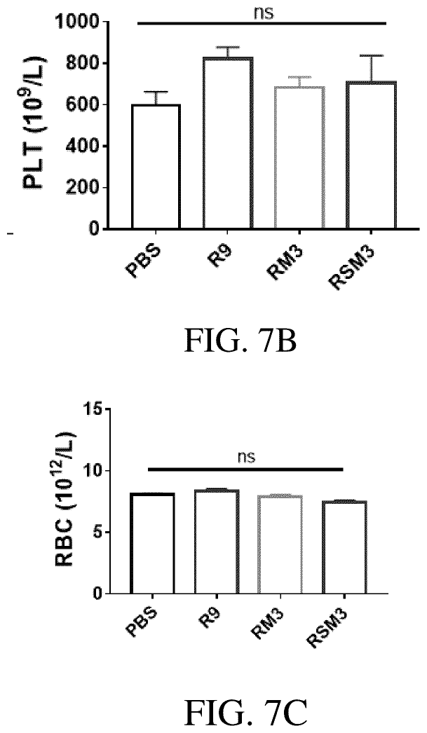

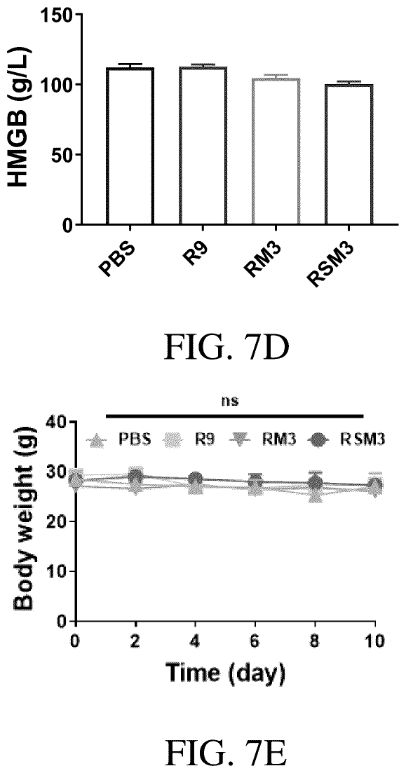

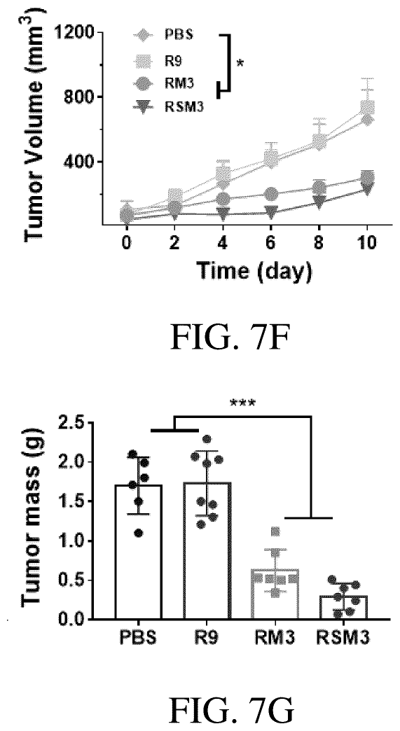

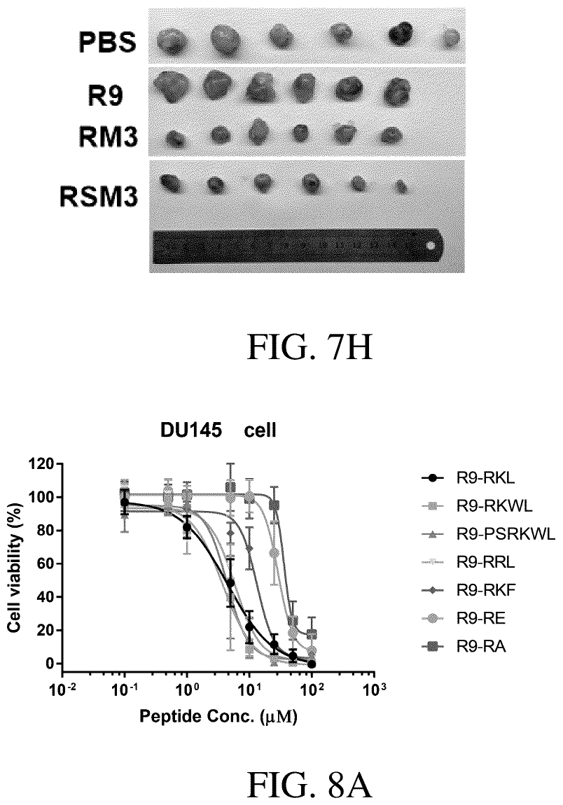

Referring to FIGS. 7A to 7H, (A) drug safety assessment in vivo: C57BL/C mice raised for 7 weeks were intraperitoneally injected with a dose of 50 mg/kg (R9, RM3, RSM3) once, after 24 hours, the mice were sacrificed and the blood of the mice was taken to measure indexes such as white blood cells, platelets, red blood cells, and hemoglobin, to analyze toxicity of the drug to normal animals.

(B) Anti-tumor experiment in vivo: a prostate tumor-bearing mouse model is selected, the specific steps to construct the animal model are as follows: PC3 prostate cancer cells with high expression of METTL3 were cultured to a logarithmic growth phase, the PC3 prostate cancer cells were digested, collected, and prepared into a cell suspension with a concentration of 1×10⁷ cells/mL. 4 to 6 week-old male and female BALB/c nude mice were taken, and were injected with 100 μL of cell suspension into the back of the hind limbs, to establish a tumor model. After a diameter of the mouse tumor grows to about 0.5 cm, the mice were randomly divided into groups according to the experimental requirements: PBS, R9, RM3, RSM3, etc. Peritumoral administration is performed on the mice, once every two days, for two consecutive weeks. After administration, the growth of the mice was observed for six consecutive weeks, the changes in tumor volume and weight of the mice were measured, the survival state of the mice was monitored, and the tumor inhibition rate and survival rate were analyzed.

In FIGS. 7A to 7H, FIGS. 7A to 7D are blood index data.

FIGS. 7E to 7H show the body weight, tumor volume changes, tumor mass and images of tumor model mice.

It can be seen from the results of FIG. 7A to 7H that:

Referring to FIGS. 7A to 7H, in terms of white blood cells (WBC) in the blood, the polypeptide inhibitors RM3 and RSM3 slightly increased this index, but the index was within the normal range, compared with the cell-penetrating peptide R9 and the control group PBS;

Referring to FIG. 7B, in terms of platelets (PLT), the polypeptide inhibitors RM3 and RSM3 had no significant effect on this index, compared with the cell-penetrating peptide R9 and the control group PBS;

Referring to FIG. 7C, in terms of red blood cells (RBC), the polypeptide inhibitors RM3 and RSM3 had no significant effect on this index, compared with the cell-penetrating peptide R9 and the control group PBS;

Referring to FIG. 7D, in terms of hemoglobin (HMGB), the polypeptide inhibitors RM3 and RSM3 had a certain reducing effect on this index, this index was within the normal range;

Referring to FIG. 7E, in terms of body weight changes, the PBS group, the cell-penetrating peptide R9, and the polypeptide inhibitor groups RM3 and RSM3 had no significant effect on the body weight of mice, which indicates that these treatment groups had no obvious toxic side effects;

Referring to FIG. 7F, in terms of tumor volume changes, polypeptide inhibitors RM3 and RSM3 can significantly inhibit the increase in tumor volume, which indicates that the polypeptide inhibitors can effectively inhibit the tumor growth;

Referring to FIG. 7G, the tumor mass was used to further determine the therapeutic effect of the polypeptide, the mass of the tumor mass in mice treated with polypeptide inhibitors RM3 and RSM3 was significantly less than that in the cell-penetrating peptide R9 group and the PBS treatment The result further shows that RM3 and RSM3 have good therapeutic effects and can inhibit group. the tumor growth;

Referring to FIG. 7H, the changes in tumor volume can confirm the results of FIGS. 7F and 7G;

Based on the above analysis, it can be seen that METTL3 polypeptide inhibitors have the effect of inhibiting the growth of prostate tumors, the stapled polypeptide inhibitor RSM3 is more effective.

It will be apparent to those skilled in the art that the present disclosure is not limited to the details of the exemplary embodiments described above, and that the present disclosure can be implemented in other specific forms without departing from the spirit or essential features of the present disclosure. Therefore, the embodiments should be considered exemplary and non-limited in all respects, and the scope of the present disclosure is defined by the appended claims rather than the foregoing description, and it is intended that all variations falling within the meaning and range of equivalent elements of the claims be included in the present disclosure. Any reference numeral in the claims should not be considered as limiting the claim to which it relates.

1. A polypeptide, comprising:
(I), an amino acid sequence as shown in any one of SEQ ID No.1, SEQ ID No.4, and SEQ ID No.5; or
(II), an amino acid sequence obtained by substituting, deleting, or adding one or more amino acids to the amino acid sequence of (I), and having a same function as the amino acid sequence of (I); or
(III), an amino acid sequence with greater than 80% similarity to the amino acid sequence of (I) or (II); or (IV), a cell-penetrating polypeptide obtained by combining the polypeptide of (I), (II), or (III) with a cell-penetrating peptide; or
(V), a stapled peptide obtained by stapling the polypeptide of (I), (II), or (IV).

2. The polypeptide according to claim 1, wherein the cell-penetrating peptide is any one of R9 cell-penetrating peptide, TAT cell-penetrating peptide, iRGD, and Penetratin cell-penetrating peptide.

3. The polypeptide according to claim 1, wherein the polypeptide is a stapled peptide obtained by stapling the polypeptide with the amino acid sequence as shown in any one of SEQ ID No.1, SEQ ID No.4, and SEQ ID No.5, or a stapled peptide obtained by replacing at least one amino acid in the amino acid sequence as shown in SEQ ID No.1 with a reactive side chain non-natural amino acid.

4. The polypeptide according to claim 1, wherein the polypeptide comprises an amino acid sequence as shown in SEQ ID No.2.

5. The polypeptide according to claim 4, wherein the polypeptide is a stapled peptide obtained by connecting 2nd and 9th cysteine in the polypeptide of the amino acid sequence as shown in SEQ ID No.2 with 4,4-bis(bromomethyl)biphenyl.

6. The polypeptide according to claim 2, wherein the cell-penetrating polypeptide comprises an amino acid sequence as shown in any one of SEQ ID No.3, and SEQ ID No.8 to 18.

7. The polypeptide according to claim 6, wherein the polypeptide is a stapled peptide obtained by connecting 11th and 17th cysteine in the polypeptide of the amino acid sequence as shown in SEQ ID No.3 with 4,4-bis(bromomethyl)biphenyl.

8. A preparation method of the polypeptide according to claim 1, comprising calculating and weighing required amino acids, dissolving the amino acids using AM resin, placing the amino acids in a synthesis bottle, setting up synthesis of the amino acids according to operations of a fully automatic synthesizer, taking out the AM resin, obtaining the polypeptide by cutting and precipitating the AM resin, and purifying the polypeptide.

9. Use of the polypeptide according to claim 1, comprising preparing a drug for treating cancer using the polypeptide, or inhibiting growth and/or proliferation of tumor cells using the polypeptide.

10. The use of the polypeptide according to claim 9, wherein the drug is used to treat one or more diseases of liver cancer, prostate cancer, thyroid tumor, leukemia, pancreatic cancer, colorectal cancer, lung cancer, glioblastoma, breast cancer, bladder cancer, gastric cancer, pancreatic cancer, osteosarcoma, oral squamous cell carcinoma, melanoma, ovarian cancer, head and neck squamous cell carcinoma, skin squamous cell carcinoma, and nasopharyngeal carcinoma.

11. The use of the polypeptide according to claim 10, wherein a dosage form of the drug is injection or oral preparation;
the drug is a water solution or hydrogel dispersed with the polypeptide, or the drug is a liposome, microsphere capsule, or micelle encapsulating the polypeptide, or the polypeptide constitutes the liposome, the microsphere capsule, or the micelle to form the drug in a form of shell.

12. The use of the polypeptide according to claim 11, wherein the oral preparation is a pill, tablet, capsule, or granule.

13. The use of the polypeptide according to claim 9, wherein the tumor cells are one or more of human osteosarcoma cells, human hepatoblastoma cells, human prostate cancer cells, colorectal cancer cells, human malignant melanoma, human cervical cancer cells, human alveolar basal epithelial cells, human chronic myeloid leukemia cells, and ovarian adenocarcinoma cells.
